(19) United States
(12) Patent Application Publication
(10) Pub. No.: US 2026/0108500 A1
(43) Pub. Date: Apr. 23, 2026

(54) OPHTHALMIC FORMULATIONS

(71) Applicant: PELTHOS THERAPEUTICS INC., DURHAM, NC (US)

(72) Inventors: Eric D. LANG, Plainsboro, NJ (US); Simon Paul CHANDLER, Warwick, RI (US); Deshou JIANG, Belle Mead, NJ (US); Richard MALAMUT, Philadelphia, PA (US)

(73) Assignee: PELTHOS THERAPEUTICS INC., DURHAM, NC (US)

(21) Appl. No.: 19/365,360

(22) Filed: Oct. 22, 2025

Related U.S. Application Data
(60) Provisional application No. 63/710,673, filed on Oct. 23, 2024.
(60) Provisional application No. 63/747,864, filed on Jan. 21, 2025.

(51) Int. Cl. A61K 31/426; A61K 9/06; A61K 47/10; A61K 47/26; A61K 47/38; A61P 27/04

(52) U.S. Cl. CPC A61K 31/426; A61K 9/06; A61K 47/10; A61K 47/26; A61K 47/38; A61P 27/04

(57) ABSTRACT

The present invention relates to ophthalmic formulations comprising 2-{[3-(5-chloro-2-{2-chloro-5-fluoro-4-[(1,3-thiazol-4-yl)sulfamoyl]phenoxy}phenyl)propyl]amino}acetamide or a pharmaceutically acceptable salt thereof, to methods for preparation thereof, and uses thereof for treatment of various eye conditions (e.g., pain).

### CROSS REFERENCE TO RELATED APPLICATIONS

This application claims the benefit of U.S. Provisional Patent Application No. 63/710,673, filed Oct. 23, 2024 and U.S. Provisional Patent Application No. 63/747,864, filed Jan. 21, 2025, both of which are incorporated herein by reference in their entirety.

### FIELD OF THE INVENTION

The present invention relates to ophthalmic formulations comprising 2-{[3-(5-chloro-2-{2-chloro-5-fluoro-4-[(1,3-thiazol-4-yl)sulfamoyl]phenoxy}phenyl)propyl]amino}acetamide or a pharmaceutically acceptable salt thereof, to methods for preparation thereof, and to uses thereof for treatment of various eye conditions (e.g., pain).

### BACKGROUND OF THE INVENTION

Compound 2-{[3-(5-chloro-2-{2-chloro-5-fluoro-4-[(1,3-thiazol-4-yl)sulfamoyl]phenoxy}phenyl)propyl]amino}acetamide) has been found to be effective for use in the treatment of various eye conditions, e.g., pain, with reduced or zero side effects.

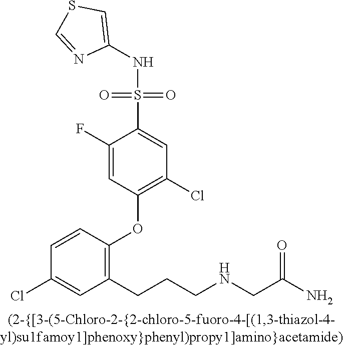 [SMILES: NC(=O)CNCCCc1cc(Cl)ccc1Oc1cc(F)c(S(=O)(=O)Nc2cscn2)cc1Cl]

2-{[3-(5-chloro-2-{2-chloro-5-fluoro-4-[(1,3-thiazol-4-yl)sulfamoyl]phenoxy}phenyl)propyl]amino}acetamide and its salts (e.g., hydrochloride salt) are low in solubility in both aqueous and oil phases. There is a need to provide desired ophthalmic formulations of the compound with high concentrations that may provide a therapeutic effective amount of the active ingredient for the treatment of various eye conditions.

### SUMMARY OF THE INVENTION

The present invention provides a pharmaceutical ophthalmic formulation comprising an active ingredient and a pharmaceutically acceptable buffer, osmolality adjuster, viscosity adjuster, solubilizer, or carrier, or any combination thereof, wherein the active ingredient is Compound A,

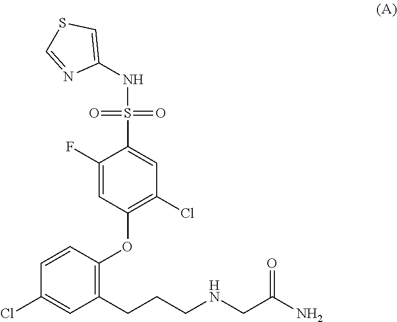 [SMILES: NC(=O)CNCCCc1cc(Cl)ccc1Oc1cc(F)c(S(=O)(=O)Nc2cscn2)cc1Cl]

or a pharmaceutically acceptable salt thereof, wherein the concentration of Compound A, or a pharmaceutically acceptable salt thereof, is about 1 mg/mL-200 mg/mL.

In some embodiments, the active ingredient of the ophthalmic formulation is a hydrochloride salt of Compound A

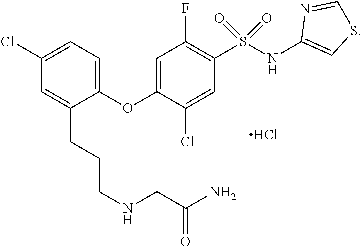 [SMILES: NC(=O)CNCCCc1cc(Cl)ccc1Oc1cc(F)c(S(=O)(=O)Nc2cscn2)cc1Cl ; Cl]

In some embodiments, the ophthalmic formulation of the invention comprises about 10 mg/mL a HCl salt of Compound A, about 12% (w/w) HPβCD, about 0.5%-4% (w/w) polysorbate 80, about 1.5% (w/w) CMC-Na (or about 3% (w/w) HPMC), and about 0.1% (w/w) NaCl in 20 mM phosphate buffer.

In some embodiments, the ophthalmic formulation of the invention comprises 20 mg/mL a HCl salt of Compound A, about 12% (w/w) HPβCD, about 4% (w/w) polysorbate 80, about 1.5% (w/w) CMC-Na (or about 3% (w/w) HPMC), and about 0.1% (w/w) NaCl in 20 mM phosphate buffer. In some embodiments, the ophthalmic formulation of the invention comprises about 15 mg/mL a HCl salt of Compound A, about 12% (w/w) HPβCD, about 4% (w/w) polysorbate 80, about 1.5% (w/w) CMC-Na (or about 3% (w/w) HPMC), and about 0.1% (w/w) NaCl in 20 mM phosphate buffer.

In some embodiments, the ophthalmic formulation of the invention comprises 12.5 mg/mL a HCl salt of Compound A, about 12% (w/w) HPβCD, about 4% (w/w) polysorbate 80, about 1.5% (w/w) CMC-Na (or about 3% (w/w) HPMC), and about 0.1% (w/w) NaCl in 20 mM phosphate buffer.

In some embodiments, the present invention provides a pharmaceutical ophthalmic formulation comprising about 10 mg/mL Compound A or a HCl salt of Compound A, about 12% (w/w) HPβCD, about 4% (w/w) polysorbate 80, and about 3% (w/w) HPMC in 20 mM phosphate buffer.

In some embodiments, the present invention provides a pharmaceutical ophthalmic formulation comprising about 12.5 mg/mL Compound A or a HCl salt of Compound A, about 12% (w/w) HPβCD, about 4% (w/w) polysorbate 80, and about 3% (w/w) HPMC in 20 mM phosphate buffer.

In some embodiments, the present invention provides a pharmaceutical ophthalmic formulation comprising about 20 mg/mL Compound A or a HCl salt of Compound A, about 12% (w/w) HPβCD, about 4% (w/w) polysorbate 80, and about 3% (w/w) HPMC in 20 mM phosphate buffer.

In some embodiments, the present invention provides a pharmaceutical ophthalmic formulation comprising about 15 mg/mL Compound A or a HCl salt of Compound A, about 12% (w/w) HPβCD, about 4% (w/w) polysorbate 80, and about 3% (w/w) HPMC in 20 mM phosphate buffer.

In another aspect, the present invention provides a pharmaceutical in situ gel ophthalmic formulation comprising (a) about 50 mg/mL Compound A or a HCl salt of Compound A, about 22.5% Poloxamer 407, about 2.1% Poloxamer 188, about 0.5% HPMC in water; or (b) about 50 mg/mL Compound A or a HCl salt of Compound A, about 21.0% Poloxamer 407, about 1.9% Poloxamer 188, about 0.5% HPMC, 0.9% NaCl in water.

In another aspect, the present invention provides a method for treating or alleviating an eye condition comprising administering to a subject in need thereof a therapeutically effective amount of a pharmaceutical ophthalmic formulation of the invention as described anywhere herein.

In some embodiments, the eye condition is dry eye pain, chronic ocular surface pain, corneal neuralgia, corneal neuropathy, corneal neuropathic pain, chronic post-surgical corneal, ocular and/or scleral pain, Stevens-Johnson Syndrome, Sjogren's Syndrome or any auto-immune disease with ocular pain, moderate to severe primary open-angle glaucoma, corneal abrasion/ocular mechanical injury, contact lens pain, corneal ulcers, a pain caused by infections, ectropion, entropion, acute closed angle glaucoma, allergic conjunctivitis, blepharitis, intravitreal injections, chalazion, or pain from glaucoma shunt surgery or failed shunt, or an acute ocular injury or a chronic pain associated with dry eye disease.

These and other aspects of the invention will be appreciated from the ensuing descriptions of the figures and detailed description of the invention.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 depicts a representative graph of the concentration over time of 20 mg/mL CT2000 ophthalmic solution that passed through an excised rabbit cornea. Four experiments are shown (PO1 through PO4) and the mean curve added.

FIG. 2 depicts the concentrations of 10 mg/ml, 15 mg/mL and 20 mg/mL CT2000 ophthalmic solution that are retained within the tissue for both excised rabbit cornea and excised rabbit sclera.

FIG. 3 depicts Draize ocular irritation scores on day 1 of the study. The Draize scoring system gives a score out of 80 for cornea, a score out of 10 for the iris and a score out of 20 for conjunctiva. Thus, the total ocular Draize score is out of a total of 110.

FIG. 4 depicts Draize ocular irritation scores on day 6 of the study.

FIG. 5A and FIG. 5B depict animal body weights changes in the study.

FIGS. 6A-6D depict that 15 mg/mL CT2000 showed a cumulative effect which led to a statistically significant improvement in paw wipes.

FIG. 7 depicts examples of corneal staining for Day 1 and Day 4 of dosing in mouse BAC dry eye studies.

FIG. 8 depicts a scheme for a rabbit acute capsaicin study.

FIG. 9 depicts that in the study performed with 20 mg/mL CT2000, CT2000 was able to reduce ocular pain within 15 mins of administration of capsaicin and continued to show efficacy through 60 minutes following a days (QID) administration.

FIG. 10 depicts that in benzalkonium chloride dry eye studies, CT2000 reduced the frequency of paw wipes within a single day of administration and showed cumulative efficacy over the time.

FIG. 11 shows the in-situ gel formulations F1 and F2 and their delivery through the sclera. The data were similar for the cornea but much lower due to the difference in tissue mass between sclera (large) and cornea (small).

FIG. 12 shows retention of the drug at 3 hours after a single administration in cornea and sclera.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

The present invention provides a pharmaceutical ophthalmic formulation comprising 2-{[3-(5-chloro-2-{2-chloro-5-fluoro-4-[(1,3-thiazol-4-yl)sulfamoyl]phenoxy}phenyl)propyl]amino}acetamide, or a pharmaceutically acceptable salt thereof. The pharmaceutical ophthalmic formulation of the invention is unexpectedly stable and contains a high concentration of the active ingredient.

In one aspect, the present invention provides a pharmaceutical ophthalmic formulation comprising an active ingredient and a pharmaceutically acceptable buffer, osmolality adjuster, viscosity adjuster, solubilizer, or carrier, or any combination thereof, wherein the active ingredient is Compound A,

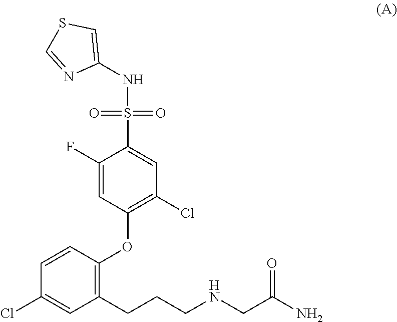 [SMILES: NC(=O)CNCCCc1cc(Cl)ccc1Oc1cc(F)c(S(=O)(=O)Nc2cscn2)cc1Cl]

or a pharmaceutically acceptable salt thereof,

In some embodiments, the concentration of Compound A, or a pharmaceutically acceptable salt thereof, is about 1 mg/mL-200 mg/mL.

In other embodiments, the concentration of Compound A, or a pharmaceutically acceptable salt thereof (e.g., a hydrochloride salt), in the ophthalmic formulation of the invention is from about 1 mg/mL to about 50 mg/mL, from about 50 mg/mL to about 100 mg/mL, from about 1 mg/mL to about 20 mg/mL, from about 20 mg/mL to about 100 mg/mL, from about 20 mg/mL to about 150 mg/mL, from about 20 mg/mL to about 200 mg/mL, from about 20 mg/mL to about 300 mg/mL, from about 20 mg/mL to about 450 mg/mL, from about 20 mg/mL to about 500 mg/mL, from about 20 mg/mL to about 750 mg/mL, or/and from about 20 mg/mL to about 1000 mg/mL.

In some embodiments, the concentration of Compound A, or a pharmaceutically acceptable salt thereof (e.g., a hydrochloride salt), in the ophthalmic formulation of the invention is from about 1.0 mg/mL to about 10 mg/mL; from about 5.0 mg/mL to about 10 mg/mL; from about 10 mg/mL to about 100 mg/mL; from about 10 mg/mL to about 70 mg/mL; from about 10 mg/mL to about 60 mg/mL; from about 10 mg/mL to about 50 mg/mL; from about 20 mg/mL to about 100 mg/mL; from about 20 mg/mL to about 70 mg/mL; from about 20 mg/mL to about 50 mg/mL; from about 30 mg/mL to about 100 mg/mL; from about 30 mg/mL to about 70 mg/mL; from about 30 mg/mL to about 50 mg/mL; from about 40 mg/mL to about 100 mg/mL; from about 40 mg/mL to about 70 mg/mL; from about 50 mg/mL to about 100 mg/mL; from about 50 mg/mL to about 70 mg/mL; from about 60 mg/mL to about 100 mg/mL; from about 70 mg/mL to about 100 mg/mL; or from about 90 mg/mL to about 100 mg/mL.

In some embodiments, the concentration of Compound A, or a pharmaceutically acceptable salt thereof (e.g., a hydrochloride salt), in the ophthalmic formulation of the invention is about 1 mg/mL, 1.5 mg/mL, 2 mg/mL, 2.5 mg/mL, 3 mg/mL, 3.5 mg/mL, 4 mg/mL, 4.5 mg/mL, 5 mg/mL, 5.5 mg/mL, 6 mg/mL, 6.5 mg/mL, 7 mg/mL, 7.5 mg/mL, 8 mg/mL, 8.5 mg/mL, 9 mg/mL, 9.5 mg/mL, 10 mg/mL, 10.5 mg/mL, 11 mg/mL, 11.5 mg/mL, 12 mg/mL, 12.5 mg/mL, 13 mg/mL, 13.5 mg/mL, 14 mg/mL, 14.5 mg/mL, 15 mg/mL, 14.5 mg/mL, 16 mg/mL, 16.5 mg/mL, 17 mg/mL, 17.5 mg/mL, 18 mg/mL, 18.5 mg/mL, 19 mg/mL, 19.5 mg/mL, 20 mg/mL, 20.5 mg/mL, 21 mg/mL, 21.5 mg/mL, 22 mg/mL, 22.5 mg/mL, 23 mg/mL, 23.5 mg/mL, 24 mg/mL, 24.5 mg/mL, 25 mg/mL, 25.5 mg/mL, 26 mg/mL, 26.5 mg/mL, 27 mg/mL, 27.5 mg/mL, 28 mg/mL, 28.5 mg/mL, 29 mg/mL, 29.5 mg/mL, 30 mg/mL, 35 mg/mL, 40 mg/mL, 45 mg/mL, or 50 mg/mL.

In some embodiments, the concentration of Compound A, or a pharmaceutically acceptable salt thereof (e.g., a hydrochloride salt), in the injectable formulation of the invention is about 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 110 mg/mL, 125 mg/mL, 150 mg/mL, 175 mg/mL, 200 mg/mL, 225 mg/mL, 250 mg/mL, 275 mg/mL, 300 mg/mL, 325 mg/mL, 350 mg/mL, 375 mg/mL, or 400 mg/mL.

In other embodiments, the concentration of Compound A, or a pharmaceutically acceptable salt thereof (e.g., a hydrochloride salt), in the ophthalmic formulation of the invention is about 60 mg/mL, 70 mg/mL, 80 mg/mL, 90 mg/mL, 100 mg/mL, 110 mg/mL, 120 mg/mL, 125 mg/mL, 200 mg/mL, 250 mg/mL, 300 mg/mL, 450 mg/mL, 500 mg/mL, 600 mg/mL, 900 mg/mL, or 1000 mg/mL.

In some embodiments of the ophthalmic formulation of the invention, the active ingredient is Compound A in its free base form. In other embodiments, Compound A can be in the form of a pharmaceutically acceptable salt. Accordingly, in some embodiments, the active ingredient is a pharmaceutically acceptable salt of Compound A. In some embodiments, the pharmaceutically acceptable salt is of Compound A and an acid selected from the group consisting of acetic acid, benzoic acid, citric acid, dichloroacetic acid, fumaric acid, HCl, HBF₄, succinic acid, L-tartaric acid, maleic acid, H₂SO₄, HBr, H₃PO₄, 1,5-naphthalenedisulfonic acid, benzenesulfonic acid, dibenzoyl-d-tartaric acid, oxalic acid, R-mandelic acid, salicylic acid, methanesulfonic acid, p-toluenesulfonic acid, s-camphorsulfonic acid, TFA, ethanesulfonic acid. In some embodiments, the pharmaceutically acceptable salt is of Compound A and an acid selected from the group consisting of hydrochloride acid, hydrobromic acid, and sulfuric acid. In some embodiments, the active ingredient of the ophthalmic formulation is a HCl salt of Compound A

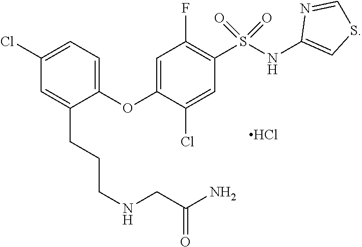 [SMILES: NC(=O)CNCCCc1cc(Cl)ccc1Oc1cc(F)c(S(=O)(=O)Nc2cscn2)cc1Cl ; Cl]

As used herein, the terms “Compound A”, “Compound A free base”, “Compound A in its free base form”, and “free base of Compound A” are used interchangeably. In some embodiments, such terms are used to distinguish from the term “salt of Compound A”, “salt form of Compound A” or “Compound A's HCl salt”.

In some embodiments, as used herein, the term “CC8464” is meant for Compound A, while the term “CC8464 HCl” is meant for a “HCl salt of Compound A” or “hydrochloride salt of Compound A”.

As used herein, Compound A or a hydrochloride salt of Compound A can be prepared by any methods known in the art, e.g., as those described in PCT Patent Publication No. WO 2015/038533.

In some embodiments, the pharmaceutical ophthalmic formulation is an ophthalmic solution formulation (e.g., eye drop). In other embodiments, the pharmaceutical ophthalmic formulation is an ophthalmic gel formulation (e.g., in situ gel).

In some embodiments of the formulations of the invention, the buffer is a phosphate buffer.

In other embodiments, the buffer includes, but is not limited to, a citrate, acetate, phosphate, lactate, succinate, histidine, tartrate, or maleate buffer or a sodium carbonate buffer, wherein the pH is about 4.0 to 8.5, or about 5.0 to 8.0, or about 6.0 to 8.0, or about 7.0 to 8.0.

In some embodiments, the solubilizer comprises hydroxypropyl beta cyclodextrin (HPβCD) or/and polysorbate.

In other embodiments, the solubilizer is PS20 (e.g., 5%, 10%, or 15%), Cremophor EL (1%, 2.5%, or 5%), PEG 400 (e.g., 1%), CMC Na (e.g., 1%), glycerin (e.g., 1%), SBECD (e.g., 5% or 10%), (alpha, beta, or gamma) CD, propylene glycol, ethanol, propanol, HP-γ-CD (e.g., 2%, 4%, 8%, or 10%), PS60, PS 80 (0.5%, 1%, 2%, or 4%), or HP-β-CD (e.g., 4%).

In some embodiments, the viscosity adjuster is sodium carboxymethylcellulose (CMC-Na). In other embodiments, the viscosity adjuster is hydroxypropyl methylcellulose (HPMC). In certain embodiments, the viscosity adjuster comprises sodium carboxymethylcellulose (CMC-Na) and hydroxypropyl methylcellulose (HPMC).

In some embodiments, the osmolality adjuster is sodium chloride or sucrose. In some embodiments, the osmolality adjuster is sodium chloride. In some embodiments, the osmolality adjuster is sucrose.

In some embodiments, the ophthalmic formulation comprises a HCl salt of Compound A, a phosphate buffer, a solubilizer, a viscosity adjuster, and an osmolality adjuster.

In some embodiments, the ophthalmic formulation of the invention comprises about 10 mg/mL a HCl salt of Compound A, about 12% (w/w) HPβCD, optionally 0.5% polysorbate 80, about 1.5% (w/w) CMC-Na (or about 3% (w/w) HPMC), and about 0.1% (w/w) NaCl in 20 mM phosphate buffer. In some embodiments, the ophthalmic formulation of the invention comprises about 10 mg/mL a HCl salt of Compound A, about 12% (w/w) HPβCD, about 0.5%-4% (w/w) polysorbate 80, about 1.5% (w/w) CMC-Na (or about 3% (w/w) HPMC), and about 0.1% (w/w) NaCl in 20 mM phosphate buffer. In some embodiments, the ophthalmic formulation of the invention comprises about 0.5%, 1.0%, 2.0%, or 4% (w/w) polysorbate 80. In certain embodiments, the ophthalmic formulation of the invention comprises about 10 mg/mL a HCl salt of Compound A, about 12% (w/w) HPβCD, about 4% polysorbate 80, about 1.5% (w/w) CMC-Na (or about 3% (w/w) HPMC), and about 0.1% (w/w) NaCl in 20 mM phosphate buffer.

In some embodiments, the ophthalmic formulation of the invention comprises 20 mg/mL a HCl salt of Compound A, about 12% (w/w) HPβCD, about 4% (w/w) polysorbate 80, about 1.5% (w/w) CMC-Na (or about 3% (w/w) HPMC), and about 0.1% (w/w) NaCl in 20 mM phosphate buffer. In some embodiments, the ophthalmic formulation comprises about 15 mg/mL a HCl salt of Compound A, about 12% (w/w) HPβCD, about 4% (w/w) polysorbate 80, about 1.5% (w/w) CMC-Na (or about 3% (w/w) HPMC), and about 0.1% (w/w) NaCl in 20 mM phosphate buffer.

In some embodiments of the method of the invention, the ophthalmic formulation comprises about 12.5 mg/mL a HCl salt of Compound A, about 12% (w/w) HPβCD, about 4% (w/w) polysorbate 80, about 1.5% (w/w) CMC-Na (or about 3% (w/w) HPMC), and about 0.1% (w/w) NaCl in 20 mM phosphate buffer.

In some embodiments, the present invention provides a pharmaceutical ophthalmic formulation comprising about 10 mg/mL Compound A or a HCl salt of Compound A, about 12% (w/w) HPβCD, about 4% (w/w) polysorbate 80, and about 3% (w/w) HPMC in 20 mM phosphate buffer. In certain embodiments, the formulation comprises a HCl salt of Compound A. In certain embodiments, the formulation comprises Compound A. In some embodiments, the formulation is an ophthalmic solution formulation.

In some embodiments, the present invention provides a pharmaceutical ophthalmic formulation comprising about 20 mg/mL Compound A or a HCl salt of Compound A, about 12% (w/w) HPβCD, about 4% (w/w) polysorbate 80, and about 3% (w/w) HPMC in 20 mM phosphate buffer. In certain embodiments, the formulation comprises a HCl salt of Compound A. In certain embodiments, the formulation comprises Compound A. In some embodiments, the formulation is an ophthalmic solution formulation.

In some embodiments, the present invention provides a pharmaceutical ophthalmic formulation comprising about 15 mg/mL Compound A or a HCl salt of Compound A, about 12% (w/w) HPβCD, about 4% (w/w) polysorbate 80, and about 3% (w/w) HPMC in 20 mM phosphate buffer. In certain embodiments, the formulation comprises a HCl salt of Compound A. In certain embodiments, the formulation comprises Compound A. In some embodiments, the formulation is an ophthalmic solution formulation.

In some embodiments, the present invention provides a pharmaceutical ophthalmic formulation comprising about 12.5 mg/mL Compound A or a HCl salt of Compound A, about 12% (w/w) HPβCD, about 4% (w/w) polysorbate 80, and about 3% (w/w) HPMC in 20 mM phosphate buffer. In certain embodiments, the formulation comprises a HCl salt of Compound A. In certain embodiments, the formulation comprises Compound A. In some embodiments, the formulation is an ophthalmic solution formulation.

In another aspect, the present invention provides a pharmaceutical in situ gel ophthalmic formulation comprising about 50 mg/mL Compound A or a HCl salt of Compound A, about 22.5% Poloxamer 407, about 2.1% Poloxamer 188, about 0.5% HPMC in water. In certain embodiments, the formulation comprises a HCl salt of Compound A. In certain embodiments, the formulation comprises Compound A.

In another aspect, the present invention provides a pharmaceutical in situ gel ophthalmic formulation comprising about 50 mg/mL Compound A or a HCl salt of Compound A, about 21.0% Poloxamer 407, about 1.9% Poloxamer 188, about 0.5% HPMC, 0.9% NaCl in water. In certain embodiments, the formulation comprises a HCl salt of Compound A. In certain embodiments, the formulation comprises Compound A.

In another aspect, the present invention provides a method for preparing a pharmaceutical ophthalmic formulation of the invention as described here, comprising
(i) dissolving a HCl salt of Compound A in 50% (w/w) HPβCD and H₂O, followed by adding polysorbate 80 to provide a stock solution, and
(ii) mixing 200 mM phosphate buffer (pH 8.0), 5% (w/w) CMC-Na or 10% (w/w) HPMC, and 1.8% (w/w) NaCl with the stock solution of step (i), adjusting the pH of the solution to 8.0, and adding water to the solution and filtering to provide the pharmaceutical ophthalmic formulation.

It is another aspect of the present invention that the ophthalmic formulation is unexpectedly stable and contains a high load or concentration of the active ingredient, e.g., Compound A or a pharmaceutically acceptable salt thereof. It exhibits distinct advantages of long shelf life for the stability of the ophthalmic formulation and provides sustained release to reduce the frequency of dosing. In some embodiments, the formulation of the invention has a shelf life of at least 1 year, or at least 1.5 years, or at least 2 years. In some embodiments, the formulation, optionally lyophilized, can be kept at a low temperature (e.g., at about −5° C. or about −15° C.) to extend its shelf life.

In some embodiments, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the “pharmaceutically acceptable salt” or “pharmaceutically acceptable salts” refers to derivatives of Compound A wherein the parent compound is modified by converting an existing base moiety to its salt form.

In other embodiments, the pharmaceutically acceptable salt of Compound A is formed from suitable non-toxic organic or inorganic acids including, but are not limited to, inorganic acids such as hydrochloric acid, hydrobromic acid, phosphoric acid, or sulfuric acid, and perchloric acid, or with organic acids such as acetic acid, oxalic acid, maleic acid, tartaric acid, citric acid, succinic acid, or malonic acid. In some embodiments, the pharmaceutically acceptable salt is of Compound A and an acid selected from the group consisting of acetic acid, benzoic acid, citric acid, dichloroacetic acid, fumaric acid, HCl, HBF₄, succinic acid, L-tartaric acid, maleic acid, H₂SO₄, HBr, H₃PO₄, 1,5-naphthalenedisulfonic acid, benzenesulfonic acid, dibenzoyl-d-tartaric acid, oxalic acid, R-mandelic acid, salicylic acid, methanesulfonic acid, p-toluenesulfonic acid, s-camphorsulfonic acid, TFA, ethanesulfonic acid.

In some embodiments, the pharmaceutically acceptable salt of a compound of formula A is a hydrochloride, hydrobromide, or sulfate salt. In one embodiment, the pharmaceutically acceptable salt of the compound A is a hydrochloride salt.

It is another aspect of the invention that the pharmaceutical ophthalmic formulation of the present invention as described anywhere herein is effective for use in the treatment or alleviation of various eye conditions.

In a further aspect, the present invention provides a method for treating or alleviating an eye condition comprising administering to a subject in need thereof a therapeutically effective amount of a pharmaceutical ophthalmic formulation of the invention as described anywhere herein.

In some embodiments, the eye condition is dry eye pain (e.g., evaporative or aqueous-deficient dry eye pain), chronic ocular surface pain, corneal neuralgia, corneal neuropathy, corneal neuropathic pain, chronic post-surgical corneal, ocular and/or scleral pain, Stevens-Johnson Syndrome, Sjogren's Syndrome or any auto-immune disease with ocular pain, moderate to severe primary open-angle glaucoma, corneal abrasion/ocular mechanical injury, contact lens pain, corneal ulcers, a pain caused by infections, ectropion, entropion, acute closed-angle glaucoma, allergic conjunctivitis, blepharitis, intravitreal injections, chalazion, or pain from glaucoma shunt surgery or failed shunt, or an acute ocular injury or a chronic pain associated with dry eye disease.

In some embodiments, the eye condition is an acute ocular injury or a chronic pain associated with dry eye disease.

In some embodiments, the chronic post-surgical corneal, ocular and/or scleral pain includes, but is not limited to, LASIK, photorefractive keratectomy, second-eye cataract, corneal transplant, or any other refractive surgeries involving invasive corneal or scleral trauma.

In some embodiments, the auto-immune disease includes, but is not limited to, Stevens-Johnson Syndrome, Sjogren's Syndrome, Grave's Disease, Blau Syndrome, Cogan Syndrome, Leber Congenital Amaurosis, ocular cicatricial pemphigoid, ocular sarcoidosis, Stargardt's disease, or other systemic autoimmune diseases selected from multiple sclerosis, Lupus Erythromatosis, Behcets Disease, and/or Rheumatoid Arthritis.

In some embodiments the infections that cause inflammatory pain include, but are not limited to, bacterial or viral infections that lead to iritis, episcleritis, scleritis, conjunctivitis, keratitis, uveitis, or herpetic infection.

In some embodiments of the method of the invention, the ophthalmic formulation comprises about 10 mg/mL a HCl salt of Compound A, about 12% (w/w) HPβCD, about 0.5%-4% (w/w) polysorbate 80, about 1.5% (w/w) CMC-Na (or about 3% (w/w) HPMC), and about 0.1% (w/w) NaCl in 20 mM phosphate buffer. In some embodiments, the ophthalmic formulation of the invention comprises about 10 mg/mL a HCl salt of Compound A, about 12% (w/w) HPβCD, about 0.5%-4% (w/w) polysorbate 80, about 1.5% (w/w) CMC-Na (or about 3% (w/w) HPMC), and about 0.1% (w/w) NaCl in 20 mM phosphate buffer. In certain embodiments, the ophthalmic formulation of the invention comprises about 10 mg/mL a HCl salt of Compound A, about 12% (w/w) HPβCD, about 4% polysorbate 80, about 1.5% (w/w) CMC-Na (or about 3% (w/w) HPMC), and about 0.1% (w/w) NaCl in 20 mM phosphate buffer.

In some embodiments of the method of the invention, the ophthalmic formulation comprises about 12.5 mg/mL a HCl salt of Compound A, about 12% (w/w) HPβCD, about 4% (w/w) polysorbate 80, about 1.5% (w/w) CMC-Na (or about 3% (w/w) HPMC), and about 0.1% (w/w) NaCl in 20 mM phosphate buffer.

In some embodiments of the method of the invention, the ophthalmic formulation comprises 20 mg/mL a HCl salt of Compound A, about 12% (w/w) HPβCD, about 4% (w/w) polysorbate 80, about 1.5% (w/w) CMC-Na (or about 3% (w/w) HPMC), and about 0.1% (w/w) NaCl in 20 mM phosphate buffer. In some embodiments of the method of the invention, the ophthalmic formulation comprises about 15 mg/mL a HCl salt of Compound A, about 12% (w/w) HPβCD, about 4% (w/w) polysorbate 80, about 1.5% (w/w) CMC-Na (or about 3% (w/w) HPMC), and about 0.1% (w/w) NaCl in 20 mM phosphate buffer.

In some embodiments of the method of the invention, the ophthalmic formulation comprises about 10 mg/mL Compound A or a HCl salt of Compound A, about 12% (w/w) HPβCD, about 4% (w/w) polysorbate 80, and about 3% (w/w) HPMC in 20 mM phosphate buffer. In certain embodiments, the formulation comprises a HCl salt of Compound A. In certain embodiments, the formulation comprises Compound A.

In some embodiments of the method of the invention, the ophthalmic formulation comprises about 12.5 mg/mL Compound A or a HCl salt of Compound A, about 12% (w/w) HPβCD, about 4% (w/w) polysorbate 80, and about 3% (w/w) HPMC in 20 mM phosphate buffer.

In certain embodiments, the formulation comprises a HCl salt of Compound A. In certain embodiments, the formulation comprises Compound A.

In some embodiments of the method of the invention, the ophthalmic formulation comprises about 20 mg/mL Compound A or a HCl salt of Compound A, about 12% (w/w) HPβCD, about 4% (w/w) polysorbate 80, and about 3% (w/w) HPMC in 20 mM phosphate buffer. In certain embodiments, the formulation comprises a HCl salt of Compound A. In certain embodiments, the formulation comprises Compound A.

In some embodiments of the method of the invention, the ophthalmic formulation comprises about 15 mg/mL Compound A or a HCl salt of Compound A, about 12% (w/w) HPβCD, about 4% (w/w) polysorbate 80, and about 3% (w/w) HPMC in 20 mM phosphate buffer. In certain embodiments, the formulation comprises a HCl salt of Compound A. In certain embodiments, the formulation comprises Compound A.

In some embodiments of the method of the invention, the ophthalmic formulation is an in situ gel ophthalmic formulation comprising about 50 mg/mL Compound A or a HCl salt of Compound A, about 22.5% Poloxamer 407, about 2.1% Poloxamer 188, about 0.5% HPMC in water. In certain embodiments, the formulation comprises a HCl salt of Compound A. In certain embodiments, the formulation comprises Compound A.

In some embodiments of the method of the invention, the ophthalmic formulation is an in situ gel ophthalmic formulation comprising about 50 mg/mL Compound A or a HCl salt of Compound A, about 21.0% Poloxamer 407, about 1.9% Poloxamer 188, about 0.5% HPMC, 0.9% NaCl in water. In certain embodiments, the formulation comprises a HCl salt of Compound A. In certain embodiments, the formulation comprises Compound A.

As used herein, the terms “treating” or “treatment” includes preventative as well as disorder remitative treatment. The terms “reducing” and “alleviating” have their commonly understood meaning of lessening or decreasing, or delaying, or reducing, the incidence, severity or pathogenesis of a disease, disorder or condition. In one embodiment, the term treatment refers to delayed progression of, prolonged remission of, reduced incidence of, or amelioration of symptoms associated with the disease, disorder or condition. In one embodiment, the terms “treating” “reducing”, or “alleviating” refer to a reduction in morbidity, mortality, or a combination thereof, in association with the indicated disease, disorder or condition. In one embodiment, the methods of treatment disclosed herein reduce the severity of the disease, or in another embodiment, symptoms associated with the disease, or in another embodiment, reduce the number of biomarkers expressed during disease.

As used herein, the term “a therapeutically effective amount” of a compound, e.g., Compound A, or a pharmaceutically acceptable salt thereof, means an amount of the compound that is effective to prevent, alleviate, or treat an eye condition as described herein. Determination of a therapeutically effective amount is within the skill in the art. The therapeutically effective amount or dosage of a compound according to this invention can vary within wide limits and may be determined in a manner known in the art. In cases of administration of a pharmaceutically acceptable salt, dosages may be calculated as the free form. For example, when 15 mg/mL CC8464·HCl is used, the weight refers to the free from of CC8464.

The exact dose and regimen of administration of the composition will necessarily be dependent upon the therapeutic effect to be achieved and may vary with the particular formula, the route of administration, and the age and condition of the individual subject to whom the composition is to be administered.

In one embodiment, the term “administering” refers to bringing a subject, e.g., a mammal (e.g., a human or nonhuman mammal) in contact with a pharmaceutical formulation of the present disclosure.

In some embodiments, the term “about” refers to a variation of 0.0001-5% from the indicated number or range of numbers. In one embodiment, the term “about” refers to a variation of 1-10% from the indicated number or range of numbers. In one embodiment, the term “about” refers to a variation of 0.05-1% (e.g., 0.05%, 0.1%, 0.5%, or 1%) from the indicated number or range of numbers. In one embodiment, the term “about” refers to a variable of about 0.5% from the indicated number or range of numbers. In one embodiment, the term “about” refers to a variation of up to 25% from the indicated number or range of numbers.

Select abbreviations and acronyms:
sodium carboxymethylcellulose (CMC-Na)
hydroxypropyl methylcellulose (HPMC)
hydroxypropyl beta cyclodextrin (HPβCD)
polysorbate (PS)
phosphate buffer (PB)
polyethylene glycol (PEG)
sulfobutylether-β-cyclodextrin (SBECD)
cyclodextrins (CD)
poly(lactic-co-glycolic acid) (PLGA)
N-methylpyrrolidone (NMP)
PS80 is Polysorbate 80 (PS80)

Throughout this application, various embodiments of the present disclosure may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1.5 to 2.5, from 1 to 4, from 1.5 to 4.5, from 1 to 5, from 1.5 to 5.5, from 2 to 4, from 2.5 to 4.5, from 2 to 6, from 2.5 to 6, from 3 to 6 etc., as well as individual numbers within that range, for example, 1, 1.5, 2, 2.5, 3, 3.5, 4, 4.5, 5, 5.5, and 6. This applies regardless of the breadth of the range.

Whenever a numerical range is indicated herein, it is meant to include any cited numeral (fractional or integral) within the indicated range. The phrases “ranging/ranges between” a first indicated number and a second indicated number and “ranging/ranges from” a first indicated number “to” a second indicated number are used herein interchangeably and are meant to include the first and second indicated numbers and all the fractional and integral numerals therebetween.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

In the description presented herein, each of the steps of the invention and variations thereof are described. This description is not intended to be limiting and changes in the components, sequence of steps, and other variations would be understood to be within the scope of the present invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

### Example 1. Ophthalmic Formulations of Invention

TABLE 1
Ophthalmic Solution Formulations

| | | | | | Target conc. |
|---|---|---|---|---|---|
| | | | Viscosity | Osmolality | CC8464•HCl |
| No. | Buffer | Solubilizer | adjustment | Adjuster | (mg/mL) |
| 1 | 20 mM pH 8.0 | 12% HPβCD + | 1.5% CMC Na | 0.1% NaCl | 10 |
| (PT25) | PB | 0.5% Polysorbate | or | (one example) | |
| | | 80 (optional) | 3% HPMC | | |
| 2 | 20 mM pH 8.0 | 12% HPβCD + | | | 20 |
| (PT27) | PB | 4% Polysorbate | | | |
| | | 80 | | | |

### Example 2: Preparation of Formulations

Formulation PT25 (10 mg/mL CC8464-HCl in 20 mM pH 8.0 PB buffer+12% HPβCD+0.5% PS80+1.5% CMC Na+0.1% NaCl)

To prepare eye drop at 10 mg/mL at a batch size of 30 mL, following steps were conducted:
1. Stock solution (30 mg/mL in 36% HPβCD): 300 mg CC8464-HCl was dissolved in 7.2 mL 50% HPβCD and 2.8 mL H₂O. Stir the solution at 45° C. and 600 RPM for about 2 hours until the solid is completely dissolved. Then 10000 rpm homogenization for 10 min.
2. Add 3 mL 200 mM pH 8.0 PB buffer, 150 mg PS80, 9 mL 5% CMC-Na and 1.67 mL 1.8% NaCl into the stock solution, and mix the solution well. Adjust the pH of the solution to 8.0, and then add water to the solution to 30 mL.
3. The solution was filtered through a 0.2 μm PES filter and packed into 2R vials.
   Formulation PT27 (20 mg/mL CC8464-HCl in 20 mM pH 8.0 PB buffer+12% HPβCD+4% PS80+3% HPMC)

To prepare eye drop at 20 mg/mL at a batch size of 30 mL, following steps were conducted:
1. Stock solution (60 mg/mL in 36% HPβCD): 600 mg CC8464-HCl was dissolved in 7.2 mL 50% HPβCD and 2.8 mL H₂O. Stir the solution at 45° C. and 600 RPM for about 2 hours until the solid is completely dissolved. Then 10000 rpm homogenization for 10 min.
2. Add 3 mL 200 mM pH 8.0 PB buffer, 1.2 g PS80, and 9 mL 10% HPMC into the stock solution, and mix the solution well. Adjust the pH of the solution to 8.0, and then add water to the solution to 30 mL.
3. The solution was filtered through a 0.2 m PES filter and packed into 2R vials.

### Example 3: Stability of Ophthalmic Solution Formulations

TABLE 2
Formulations of PT25 and PT27

| | | | | Osmolality | Target | Initial | | | | | | |
|---|---|---|---|---|---|---|---|---|---|---|---|---|
| | | | Viscosity | Adjuster | conc. | | | Conc. | Purity | Viscosity | Osmolality | Visible |
| No. | Buffer | Solubilizer | adjustment | (TBD) | (mg/mL) | Appearance | pH | (mg/mL) | (%) | (mPa · s) | (mOsm/kg) | particles |
| PT25 | 20 mM | 12% | 1.5% CMC | 0.1% NaCl | 10 | Clear | 8.02 | 9.00 | 99.32 | 9.27 | 305 | Pass |
| | pH 8.0 | HPβCD + | Na | | | | | | | | | |
| | PB | 0.5% PS80 | | | | | | | | | | |
| PT27 | 20 mM | 12% | 3% HPMC | 0.1% NaCl | 20 | Clear | 7.99 | 19.10 | 99.23 | 14.49 | 397 | Pass |
| | pH 8.0 | HPβCD + | | | | | | | | | | |
| | PB | 4% PS80 | | | | | | | | | | |

TABLE 3
Stability of PT25 and PT27

| | | | PT-25 | | | | PT-27 | | | |
|---|---|---|---|---|---|---|---|---|---|---|
| Conditions | | | | Conc. | | | | Conc. | | |
| (days) | | | Appearance | (mg/mL) | Purity % | TRS % | Appearance | (mg/mL) | Purity % | TRS % |
| T0 | | | Clear | 9.00 | 99.32 | — | Clear | 19.10 | 99.23 | — |
| 4 | C. | 14 | Clear | 9.12 | 98.90 | 0.42 | Clear | 16.96 | 98.95 | 0.28 |
| | | 14 | Clear | 8.75 | 98.90 | 0.42 | Clear | 17.28 | 98.96 | 0.27 |
| | | 21 | Clear | 9.51 | 98.91 | 0.41 | Clear | 17.68 | 98.93 | 0.30 |
| | | 21 | Clear | 9.35 | 98.93 | 0.39 | Clear | 19.05 | 98.93 | 0.30 |
| 25 | C. | 12 | Clear | 8.93 | 98.07 | 1.25 | Clear | 15.99 | 97.59 | 1.64 |
| | | 21 | Clear | 8.92 | 97.39 | 1.93 | Clear | 17.38 | 96.24 | 2.99 |
| 40 | C. | 4 | Clear | 8.87 | 98.35 | 0.97 | Clear | 16.20 | 96.03 | 3.20 |

| | | | | | | | | |
|---|---|---|---|---|---|---|---|---|
| 7 | Clear | 8.80 | 96.39 | 2.93 | Clear | 14.81 | 94.41 | 4.82 |
| 12 | Clear | 8.03 | 94.31 | 5.01 | Clear | 14.16 | 90.39 | 8.84 |

### Example 4: Ophthalmic Formulations

TABLE 4
Ophthalmic In Situ Gel Formulations of Invention

| | Detailed Formulation | | | | |
|---|---|---|---|---|---|
| | Conc. of | | | Target | Target |
| | API | | | Osmolality | Viscosity |
| No. | (mg/mL) | Vehicle | Target pH | (mOsm/kg) | (cp) |
| 1 | 20 | 20 mM PB + P407 + P188 | 8.0 | 290-310 | 10-15 |
| 2 | | 20 mM PB + P407 + (P188+) CMC-Na | | | |
| 3 | | 20 mM PB + P407 + (P188+) Polycarbophil | | | |
| 4 | | 20 mM PB + P407 + (P188+) HPMC | | | |
| 5 | | 20 mM PB + P407 + (P188+) HA-Na | | | |

TABLE 5
Ophthalmic In Situ Gel Formulations of Invention

| | Detailed Formulation | | |
|---|---|---|---|
| | Conc. of | | |
| No. | API (mg/mL) | Vehicle | Osmolality Adjuster |
| 6 | 50 | 18% P407 + 2% P188 in WFI | NaCl/mannitol |
| 7 | | 18% P407 + 2% P188 in WFI + 0.5% CMC-Na | |
| 8 | | 18% P407 + 2% P188 in WFI + 0.5% Polycarbophil | |
| 9 | | 18% P407 + 2% P188 in WFI + 0.5% HPMC | |
| 10 | | 18% P407 + 2% P188 in WFI + 0.1% HA-Na | |

In Tables 4 and 5, API was ball milled to reduce its particle size to nm scale prior to preparation of in situ gel formulations.

Formulation PT25

Strength: 10 mg/mL
Formulation: 10 mg/mL CC8464 in 20 mM pH 8
PB buffer +12% HBβCD + 3%
HPMC + 4% PS80

Formulation PT28

Strength: 15 mg/mL
Formulation: 15 mg/mL CC8464 in 20 mM pH 8 PB buffer + 12%
HBβCD + 3%
HPMC + 4% PS80

Formulation PT27

Strength: 20 mg/ml
Formulation: 20 mg/mL CC8464 in 20 mM pH 8 PB buffer + 12%
HBβCD + 3%
HPMC + 4% PS80

### Example 5: In Vitro Melanin Binding Assay

### Materials

Matrix: Melanin was purchased from Sigma-Aldrich. D-PBS (lx) was purchased from CORNING.

### Stop Solution

Test Compound: Acetonitrile containing tolbutamide at 250 nM and labetalol at 250 nM.
Control Compound: Acetonitrile containing tolbutamide at 250 nM and labetalol at 250 nM.

Compound information is provided in Table 6.

TABLE 6
Compounds used in the assay

| | | | Stock | Final |
|---|---|---|---|---|
| Compound | Compound | | Concentration | Concentration |
| No. | ID | MW | (mM) | (μM) |
| 1 | CC8464 | 533.42 | 10 | 1 |
| | free base | | | |
| Control | Chloroquine | 319.87 | 10 | 1 |

### Methods

### Processing Procedure of Matrix

1 mg/mL melanin was made by suspending the melanin in D-PBS (1×).

### Dilution Procedure of Test Compound and Control Compound

Working solutions (200 μM) of test compounds were prepared by diluting 2 μL of the stock solution with 98 μL DMSO.
Working solutions (200 μM) of control compounds were prepared by diluting 2 μL of the stock solution with 98 μL H2O.
Loading matrix solutions (1 μM) of test compounds were prepared by diluting 6 μL of working solutions with 1194 μL of blank matrix and 6 μL of working solutions with 1194 μL of blank D-PBS, respectively, and mixed thoroughly.
Loading matrix solutions (1 μM) of control compounds were prepared by diluting 6 μL of working solutions with 1194 μL of blank matrix and 6 μL of working solutions with 1194 μL of blank D-PBS, respectively, and mixed thoroughly.

### Procedure of Assay

Aliquots of 50 μL loading D-PBS containing test compounds or control compounds were transferred in triplicate to Sample Collection Plate, respectively. The stop solution (500 μL) was added to these TO samples of test compounds and control compounds. The plate was sealed and shaked at 800 rpm for 10 minutes. Then these TO samples were stored at 2˜8° C. pending further process along with other post-dialysis samples.

Aliquots of 300 μL of the spiked solution were loaded in triplicate to a new 96-well plate (both the drug-containing melanin solution and the drug-containing D-PBS solution). Then the plate was sealed and incubated at 37° C. for 1 hour on a rotary shaker set at 750 rpm.

After incubation, all the samples were centrifuged at 1600×g for 10 minutes to get F samples and T samples.

Aliquots of 50 μL of supernatant were removed to a new 96-well plate to get F samples (from drug-containing melanin solution, n=3) and T samples (from drug-containing D-PBS solution, n=3). Stop solution (500 L) was added to these samples. The mixture was vortexed and centrifuged at 4000 rpm for about 20 minutes. An aliquot of 100 μL of supernatant of all the samples was then removed for LC-MS/MS analysis.

The single blank samples were prepared by transferring 50 μL of blank D-PBS to each well. Then the samples were further processed by adding 500 μL of stop solution containing internal standards, following the same sample processing method as the other samples.

### Data Analysis

The results were calculated by the following equations:

$$\% Unbound = 100 \times F / T$$

$$\% Bound = 100 - \% Unbound$$

$$\% Recovery = 100 \times T / Mean of T 0$$

where F=Free peak area ratio of analyte and internal standard taken from drug-containing melanin solution after incubation; T=Total peak area ratio of analyte and internal standard taken from drug-containing D-PBS solution without melanin after incubation; and T0=The peak area ratio of analyte and internal standard taken from drug-containing D-PBS solution without melanin wells at time zero.

Results Summary for 1 mg/mL Melanin is provided in Table 7.

TABLE 7
Results Summary

| | Melanin | | | | |
|---|---|---|---|---|---|
| | % Unbound | | | % Recovery | |
| Compound ID | (n = 3) | SD | % Bound | (n = 3) | SD |
| CC8464 free base | 23.7 | 0.7 | 76.3 | 89.9 | 3.7 |
| Chloroquine | 0.3 | 0.0 | 99.7 | 97.0 | 2.9 |

Results: The free base showed moderate melanin binding ˜75% with ˜25% unbound fraction. Chloroquine was the positive control.

### Ex Vivo Cornea/Scleral Penetration Study

An ex vivo cornea/scleral penetration study was conducted to explore whether CT2000 could penetrate those ocular tissues and pass into the anterior chamber of the eye.

Using a modified Franz-type diffusion chamber, samples of rabbit cornea and sclera were tested for retention and pass-through of CT2000.

As used herein, the term “CT2000” refers to a formulation, while “CC8464” is the active ingredient in the formulation. For example, PT25/28/27 are all CT2000 ophthalmic solutions with different concentrations of the active ingredient CC8464, e.g., 15 mg/mL CT2000.

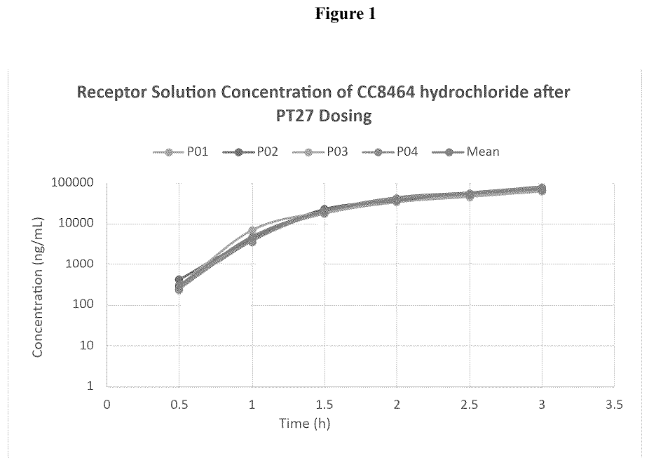

FIG. 1 depicts a representative graph of the concentration over time of 20 mg/mL CT2000 ophthalmic solution that passed through an excised rabbit cornea. Four experiments are shown (PO1 through PO4) and the mean curve added.

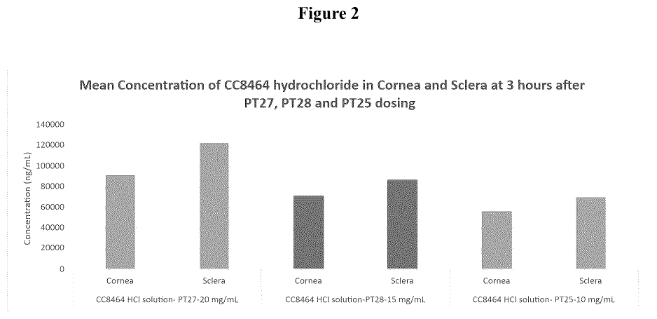

FIG. 2 depicts the concentrations of 10 mg/ml, 15 mg/mL and 20 mg/mL CT2000 ophthalmic solution that are retained within the tissue for both excised rabbit cornea and excised rabbit sclera.

### Example 6: Seven Day Rabbit Tolerability Study

An eye drop irritation study of compound CT2000 was conducted in different formulations in male NEW ZEALAND white rabbits following 7-day instilled in eyes.

The study was designed to observe ocular irritation caused by the irritation of three formulations of CT2000 following instillation into the eyes to male Dutch Belt rabbit.

TABLE 8
Study Design

| | | | | Target | Target Dose | | | |
|---|---|---|---|---|---|---|---|---|
| Group/ | # of | Test | Target | Dose | Concentration | | Dose | |
| Phase | Animal | Article | Dose Level | Volume | (mg/mL) | Vehicle | Route* | Analyte |
| G1 | 3 males | CC8464 | 4 drops (100 uL)/ | / | 10 | CC8464 HCl solution- | Instilled | CC8464 |
| | | HCl salt | each eye/day | | | PT25-10 mg/mL | in eyes | hydrochloride |
| G2 | 3 males | CC8464 | 4 drops (100 uL)/ | / | 20 | CC8464 HCl solution- | Instilled | CC8464 |
| | | HCl salt | each eye/day | | | PT27-20 mg/mL | in eyes | hydrochloride |
| G3 | 3 males | CC8464 | 4 drops (100 uL)/ | / | 15 | CC8464 HCl solution- | Instilled | CC8464 |
| | | HCl salt | each eye/day | | | PT28-15 mg/mL | in eyes | hydrochloride |

Note.
All animals will be fed before dosing.

TABLE 9
Dose Schedule

| Group | Dosage | Animal No. | Irritation test time points (hr) |
|---|---|---|---|
| G1 | 4 drops (100 uL)/each eye/day | RB1001, RB1002, RB1003 | Pre-dose, 0.5, 1, 2, 4 |
| G2 | 4 drops (100 uL)/each eye/day | RB2001, RB2002, RB2003 | Pre-dose, 0.5, 1, 2, 4 |
| G3 | 4 drops (100 uL)/each eye/day | RB3001, RB3002, RB3003 | Pre-dose, 0.5, 1, 2, 4 |

TABLE 10
Formulations

| Formulation ID | Lot: | Assay (%) | CF | Storage Condition |
|---|---|---|---|---|
| CC8464 HCl solution- PT27-20 mg/mL | FR05206-5-0924-PT-27 | 96.86 | 1.103 | 4° C. |
| CC8464 HCl solution-PT28-15 mg/mL | FR05206-5-0924-PT-28 | 97.25 | 1.099 | 4° C. |
| CC8464 HCl solution- PT25-10 mg/mL | FR05206-5-0924-PT-25 | 97.87 | 1.091 | 4° C. |

Comments: CF = FW/MW/Assay

TABLE 11
Animal Specifications

| | |
|---|---|
| Species | New Zealand White Rabbit |
| History of Dosing | Naïve animals |
| Body Weight Range | 2-2.5 kg |
| Age | >2 months |
| Sex | Male |
| Method of Identification | Unique skin tattoo on ear |
| Number of Animals for Acclimation | 10 males |
| Number of Animals for Dosing | 9 males |

TABLE 12
Administration of Dose Formulation

| | |
|---|---|
| Administration Route: | Instilled in eyes |
| Dose | For instilled in eyes, the dose |
| | formulation will be administered using |
| Administration: | pipette. The maximum dose volume |
| | will be 0.1 mL/each eye. |

Direct ophthalmoscopy was used to perform Draize ocular irritation scoring bilaterally for animals and following the scoring standard in Table 13.

TABLE 13
Draize Ocular irritation Scoring

| Ocular irritative reaction | Score |
|---|---|
| Cornea | |
| A. Opacity-degree of density (area most dense taken for reading) | |
| No opacity | 0 |
| Scattered or diffuse area, details of iris clearly visible | 1 |
| Easily discernable translucent areas, details of iris slightly obscured | 2 |
| Opalescent areas, no details of iris visible, size of pupil barely discernible | 3 |
| Opaque, iris invisible | 4 |
| B. Area of opacity | |
| Normal cornea | 0 |
| One quarter (or less) but not zero | 1 |
| Greater than one quarter, but less than half | 2 |
| Greater than half, but less than three quarters | 3 |
| Greater than three quarters, up to whole area | 4 |
| Maximum = A × B × 5 = 4 × 4 × 5 = 80 | |

Iris

| | |
|---|---|
| A. Values | |
| Normal | |
| Folds above normal, congestion, swelling, circumcorneal injection (iris s till reacting to light | 0 |
| (sluggish reactions are positive) (any or all of these or combination of any thereof) | |
| No reaction to light, hemorrhage, gross destruction (any or all of these) | 1 |
| Maximum = A × 5 = 2 × 5 = 10 | 2 |

Conjunctivae

| | |
|---|---|
| A. Hyperemia (bulbar and the palpebral conjunctiva) | |
| Vessels normal (may appear blanched to reddish pink) | 0 |
| A flushed, reddish color predominantly (confined to the palpebral conjunctiva) with some | 1 |
| perilimbal injection but (primarily confined to the lower and upper parts of the eye from the 11 | |
| to 1 o'clock and 4 to 7 o'clock positions) | |
| A flushed, reddish color predominantly (confined to the palpebral conjunctiva) with some | 2 |
| perilimbal injection but (primarily confined to the lower and upper parts of the eye from the 11 | |
| to 1 o'clock and 4 to 7 o'clock positions) | |
| Perilimbal injection covering at least 75% of the circumference of the perilimbal region | |
| Dark beefy red color with congestion of both bulbar and the palpebral | 3 |
| conjunctiva and pronounced perilimbal injection and the presence of petechiae on the | |
| conjunctiva | |

B. Chemosis (Swelling)

| | |
|---|---|
| No Swelling | 0 |
| Any swelling above normal without eversion of the lids (includes nictitating membrane). | 1 |
| Swelling generally starts in the lower cul-de-sak near the inner canthus | |
| Obvious swelling with partial eversion of lids. Misalignment of the normal approximation of | 2 |
| the lower and upper eyelids. Generally confined to the upper eyelid | |
| Swelling with lids about half closed. Partial eversion of the upper and lower eyelids | 3 |
| Swelling with lids half closed to completely closed. Pronounced eversion of the upper eyelid | 4 |
| with less pronounced eversion of the lower eyelid. It is difficult to retract the eyelids | |

C. Discharge

| | |
|---|---|
| No discharge (small amounts observed in inner and outer canthus of normal animals) | 0 |
| Few discharge, and present on the inner portion of the eye but not on the lids or hair of the | 1 |
| eyelid | |
| Discharge with moistening of the lids and hairs adjacent to eyelids | 2 |
| Discharge with moistening of the lids and hairs, and considerable area around the eye flowing | 3 |
| over the eyelids | |
| Maximum = (A + B + C)*2 = (3 + 4 + 3)*2 = 20 | |
| The maximum total score is the sum of the cornea, iris and conjunctiva. The maximum total | |
| score is 110 per eye. | |

Rabbits were dosed with the 3 formulation concentrations of 10 mg/mL, 15 mg/mL and 20 mg/mL CT2000 four times daily for 7 days.

Ocular irritation was scored based on veterinary observation and a score chart for cornea, iris and conjunctivae

### Results

On Day 1, one rabbit in 20 mg/mL arm showed minimal irritation in the iris and conjunctiva (2/10 and 2/20 respectively); and two rabbits in 10 mg/mL arm showed minimal irritation in the cornea and conjunctiva. No further findings were observed after day one of dosing.

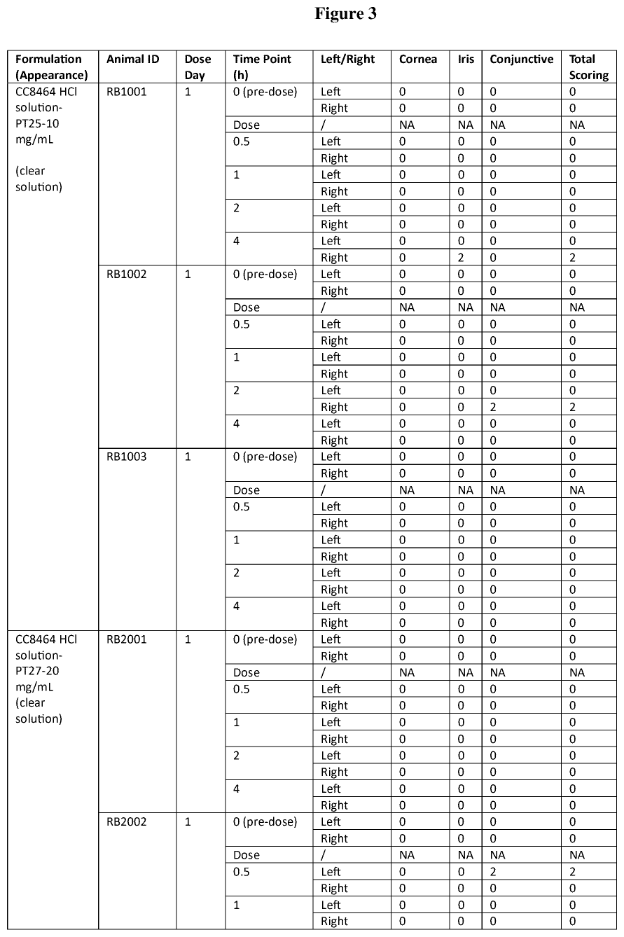

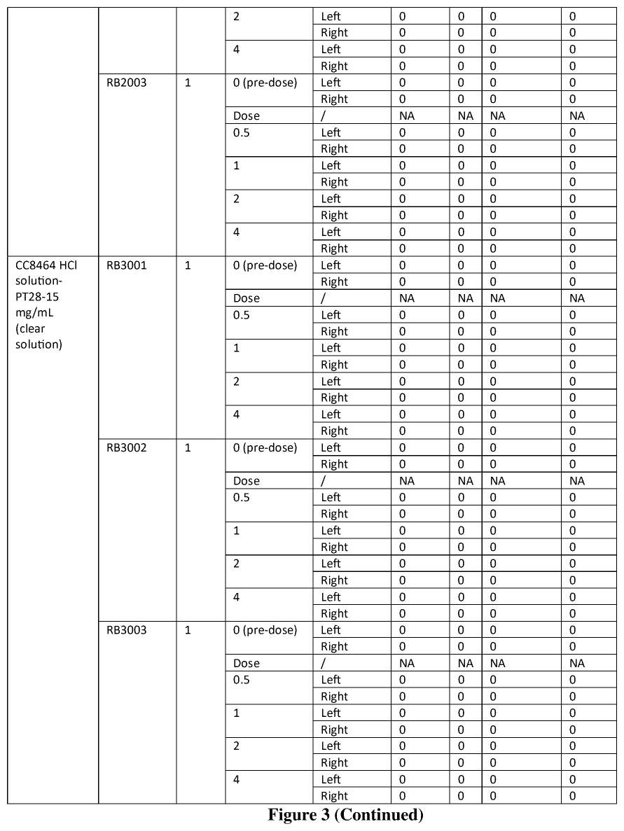

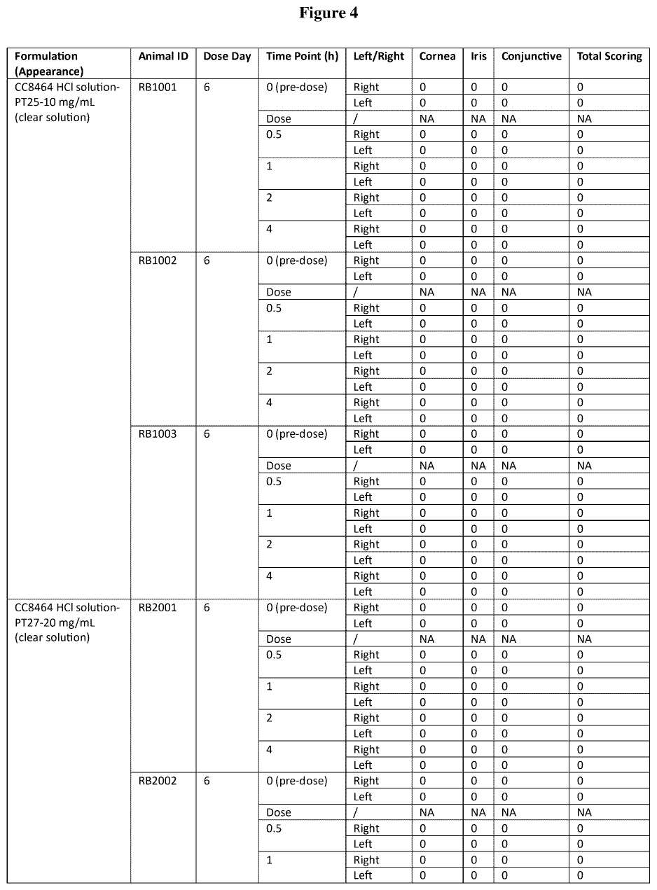

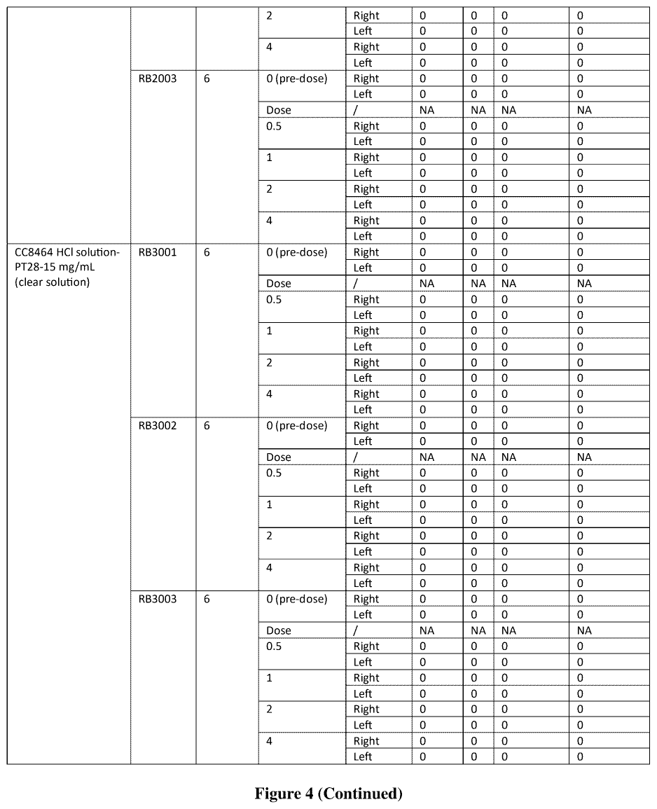

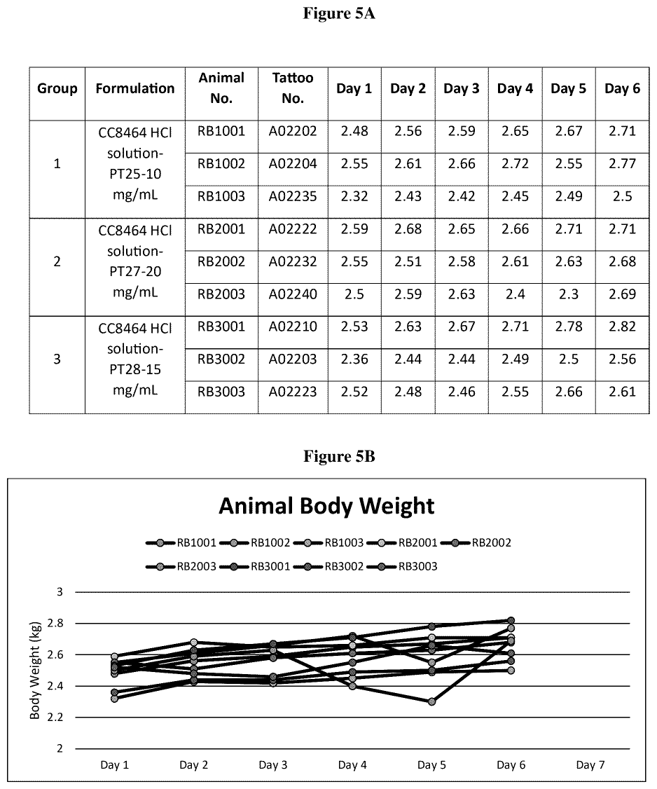

FIG. 3 depicts Draize ocular irritation scores on day 1 of the study and FIG. 4 depicts Draize ocular irritation scores on day 6 of the study. The Draize scoring system gives a score out of 80 for cornea, a score out of 10 for the iris and a score out of 20 for conjunctiva. Thus the total ocular Draize score is out of a total of 110. FIG. 5A and FIG. 5B depict animal body weights changes in the study.

Conclusions: All three dose concentrations were well tolerated over 7 days of QID dosing.

### Example 7: Mouse BAC Dry Eye Studies

Study 1: Evaluate analgesia efficacy of drug on BAC-induced dry eye model in mice.

TABLE 14
Study Design

| Group | | | |
|---|---|---|---|
| Group | | | Animal |
| number | Group name | Dosing | number |
| 1 | Model ctrl + vehicle | eye drop, QID, D8-D14 | 5 |
| 2 | BAC Model + vehicle | eye drop, QID, D8-D14 | 5 |
| 3 | BAC Model + PT-28 | eye drop, QID, D8-D14 | 5 |
| | (15 mg/ml) | | |
| 4 | BAC Model + | eye drop, BID, D8-D14 | 5 |
| | positive drug | | |
| | (Bromfenac) | | |

A repeated dose study was conducted over 7 days with daily QID dosing in the left eye of mice. These mice have induced dry eye due to benzalkonium chloride instillation over 7 days prior to CT2000 dosing as shown in Scheme 1.

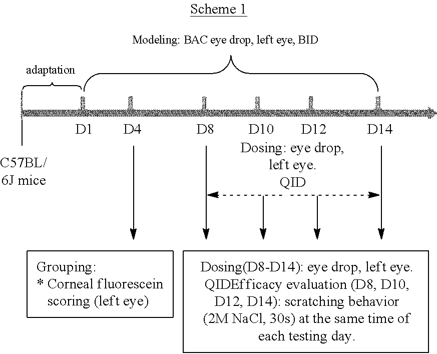

Modeling (D1-D14): BAC eye drop, on left eye, BID (8 AM, 8 PM), from D1 to D14;

Grouping (D4): Corneal fluorescein staining is imaged with slit lamp on left eye of all mice, and the staining is scored, then grouped based on the score.

Dosing of PT28 and Bromfenac (D8-D14): eye drop, left eye. QID for vehicle and PT-28 group (10:00 AM. 12:30 PM, 15:00 PM. 17:30 PM). BID for Bromfenac group (10:00 AM, 17:30 PM).

Dosing of Bromfenac 0.1% (D8-D14): eye drop, left eye. BID (10.00 am and 17.30 pm)

Tests (D8. D10, D12, D14): For drug and vehicle groups, after the second dosing (12:30 PM), mice were individually adapted in the testing cage after dosing. For Bromfenac group, after the first dosing (10:00 AM), mice were individually adapted in the testing cage after dosing. At 20 min after dosing and adaptation, 2M NaCl eye drop was delivered onto mouse left eye. Paw wipe counts were then collected for 30 seconds immediately after 2M NaCl was applied.

Corneal fluorescein staining: On D14. after last test of eye wipes

This study was performed with the PT28 15 mg/mL. Face scratching (paw wipes) behavior is a surrogate for ocular pain in this model

### Results

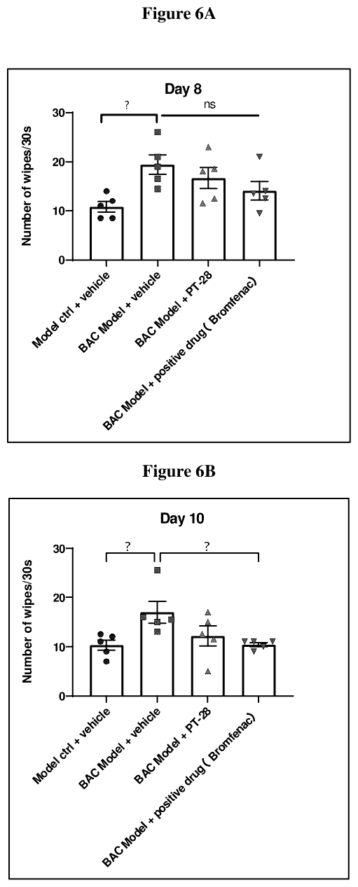

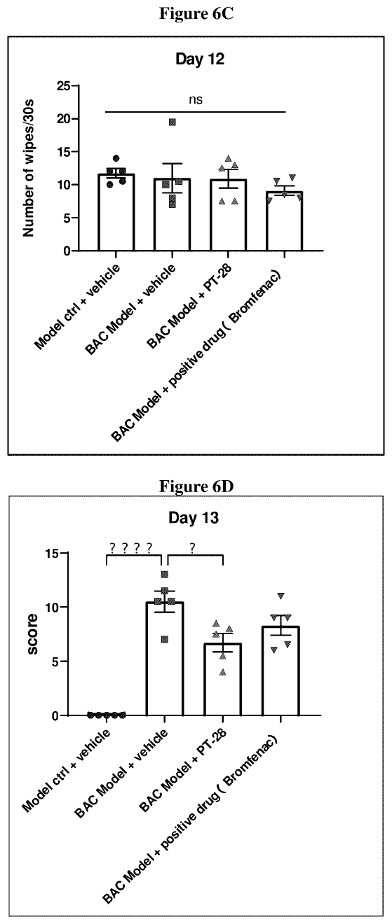

Study days 8, 10 and 12 are 2, 4 and 6 days of dosing with PT28 15 mg/mL CT2000. 15 mg/mL CT2000 showed a cumulative effect which led to a statistically significant improvement in paw wipes after 4 days of dosing. 15 mg/mL CT2000 was non-inferior to Bromfenac at 4 days. At 6 days, the mice adapted to the BAC and paw wipe number in the untreated arm (vehicle) returned to baseline (FIG. 6A, FIG. 6B, and FIG. 6C).

### Mouse BAC Dry Eye Study—Effect on Corneal Staining

After the last dose day (day 12 of the study, day 4 of CT2000 dosing) of 15 mg/mL (FIG. 6D), corneal staining was performed on the mouse dose groups

### Results

There was a statistically significant reduction in corneal staining score after 4 days of dosing with 15 mg/mL CT2000. The CT2000 vehicle did not show the reduction. This was not observed with the Bromfenac positive control.

Corneal staining is always seen with dry eye disease in humans. Reduction in staining is an FDA approved endpoint for dry eye medications when accompanied with a clinically significant reduction in a DED symptom. There is clear evidence that 15 mg/mL CT2000 can reduce both the signs (staining) and the symptoms (pain) of dry eye disease in a murine model of dry eye.

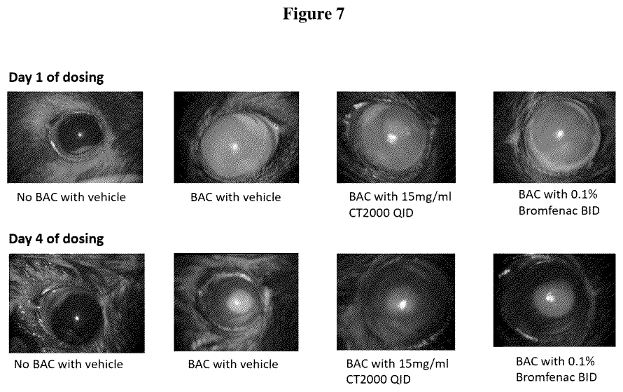

FIG. 7 provides examples of corneal staining for Day 1 and Day 4 of dosing in mouse BAC dry eye studies.

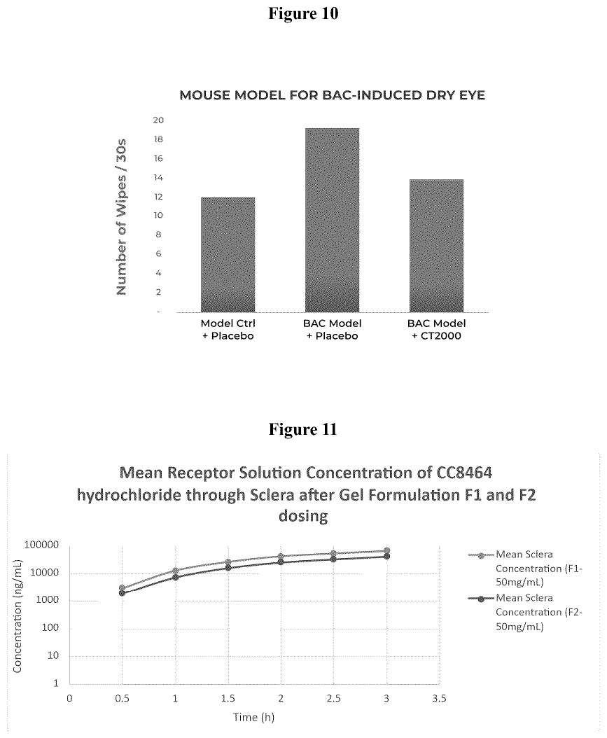

In a second study, benzalkonium chloride (“BAC”) was instilled onto mice eyes over a multiday period to create a model of dry eye disease (the study was repeated twice). BAC is a preservative that irritates the eyes and simulates dry eye disease. As with the first study, increased paw wipes over 30 seconds are a surrogate to measure ocular pain. Following the dosing of BAC, the mice were dosed with 15 mg/mL CT2000 four times per day for 7 days. CT2000 reduced the frequency of paw wipes within a single day of administration and showed cumulative efficacy over time (the analgesic effect appeared to further improve when dosed over several days). The results are summarized in FIG. 10.

Scheme 2 is for the second mouse BAC study.

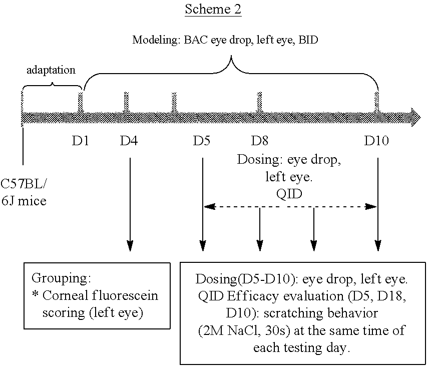

### Example 8: Rabbit Acute Capsaicin Study

Study 2: Evaluate analgesia efficacy of drug on post-surgical corneal injury model (the capsaicin model) in rabbit.

This is a rabbit capsaicin acute ocular pain study. Rabbits were treated with Capsaicin (Pepper spray) to mimic an acute ocular insult.

TABLE 15
Study Design

| Group | | | |
|---|---|---|---|
| Group | | | Animal |
| number | Group name | Dosing | number |
| A | 15 min challenge + drug A | eye drop | 5 |
| B | 60 min challenge + drug A | eye drop | 5 |

FIG. 8 depicts a scheme for a rabbit acute capsaicin study. Rabbits were dosed 4 times per eye over the day with 20 mg/mL CT2000. After the last dose of CT2000, the rabbits were challenged with capsaicin and paw wipes counted at 15 mins and 60 minutes.

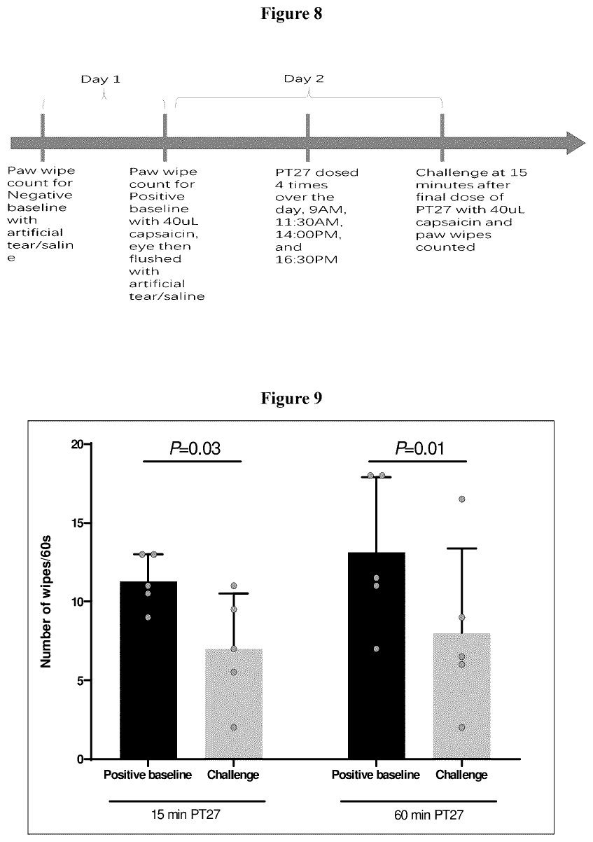

FIG. 9 depicts that in the study performed with 20 mg/mL CT2000, CT2000 was able to reduce ocular pain within 15 mins of administration of capsaicin and continued to show efficacy through 60 minutes following a days (QID) administration.

### Example 9: Ophthalmic In Situ Gel Formulations

TABLE 16
Ophthalmic In Situ Gel Formulations

| | |
|---|---|
| F1 | 50 mg/mL CC8464, 22.5% Poloxamer 407, 2.1% Poloxamer 188, 0.5% HPMC in WFI |
| F2 | 50 mg/mL CC8464, 21.0% Poloxamer 407, 1.9% Poloxamer 188, 0.5% HPMC, 0.9% NaCl in WFI |

FIG. 11 shows the in-situ gel formulations F1 and F2 and their delivery through the sclera. The data were similar for the cornea but much lower due to the difference in tissue mass between sclera (large) and cornea (small).

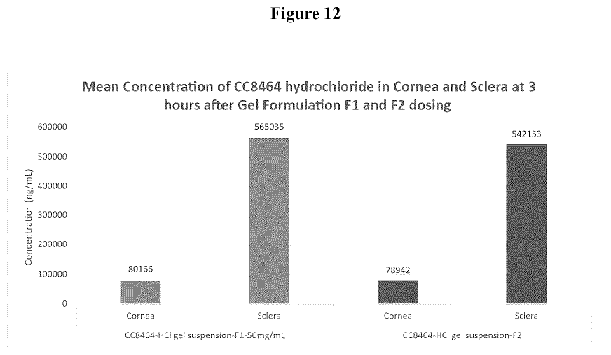

FIG. 12 shows retention of the drug at 3 hours after a single administration in cornea and sclera.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed:

1. A pharmaceutical ophthalmic formulation comprising (a) about 10 mg/mL Compound A or a HCl salt of Compound A, about 12% (w/w) HPβCD, about 4% (w/w) polysorbate 80, and about 3% (w/w) HPMC in 20 mM phosphate buffer; or (b) about 12.5 mg/mL Compound A or a HCl salt of Compound A, about 12% (w/w) HPβCD, about 4% (w/w) polysorbate 80, and about 3% (w/w) HPMC in 20 mM phosphate buffer

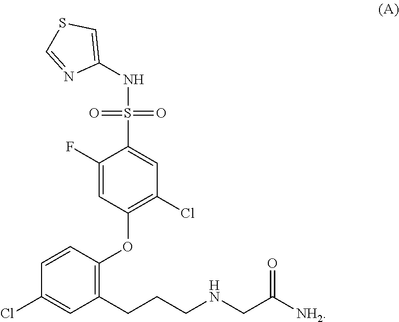 [SMILES: NC(=O)CNCCCc1cc(Cl)ccc1Oc1cc(F)c(S(=O)(=O)Nc2cscn2)cc1Cl]

2. The pharmaceutical ophthalmic formulation according to claim 1, comprising a HCl salt of Compound A

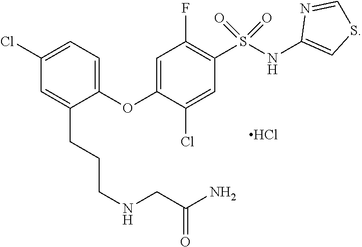 [SMILES: NC(=O)CNCCCc1cc(Cl)ccc1Oc1cc(F)c(S(=O)(=O)Nc2cscn2)cc1Cl ; Cl]

3. A method for treating or alleviating an eye condition comprising administering to a subject in need thereof a therapeutically effective amount of a pharmaceutical ophthalmic formulation according to claim 1, wherein the eye condition is dry eye pain, chronic ocular surface pain, corneal neuralgia, corneal neuropathy, corneal neuropathic pain, chronic post-surgical corneal, ocular and/or scleral pain, Stevens-Johnson Syndrome, Sjogren's Syndrome or any auto-immune disease with ocular pain, moderate to severe primary open-angle glaucoma, corneal abrasion/ocular mechanical injury, contact lens pain, corneal ulcers, a pain caused by infections, ectropion, entropion, acute closed-angle glaucoma, allergic conjunctivitis, blepharitis, intravitreal injections, chalazion, or pain from glaucoma shunt surgery or failed shunt, or an acute ocular injury or a chronic pain associated with dry eye disease.

4. The method according to claim 3, wherein chronic post-surgical corneal, ocular and/or scleral pain comprises LASIK, photorefractive keratectomy, second-eye cataract, corneal transplant, or any other refractive surgeries involving invasive corneal or scleral trauma.

5. The method according to claim 3, wherein the auto-immune disease comprises Stevens-Johnson Syndrome, Sjogren's Syndrome, Grave's Disease, Blau Syndrome, Cogan Syndrome, Leber Congenital Amaurosis, ocular cicatricial pemphigoid, ocular sarcoidosis, Stargardt's disease, or other systemic autoimmune diseases selected from multiple sclerosis, Lupus Erythromatosis, Behcets Disease, and/or Rheumatoid Arthritis.

6. The method according to claim 3, wherein the pain caused by infections comprises bacterial or viral infections leading to iritis, episcleritis, scleritis, conjunctivitis, keratitis, uveitis, or herpetic infection.

7. A pharmaceutical ophthalmic formulation comprising about 20 mg/mL Compound A or a HCl salt of Compound A, about 12% (w/w) HPβCD, about 4% (w/w) polysorbate 80, and about 3% (w/w) HPMC in 20 mM phosphate buffer

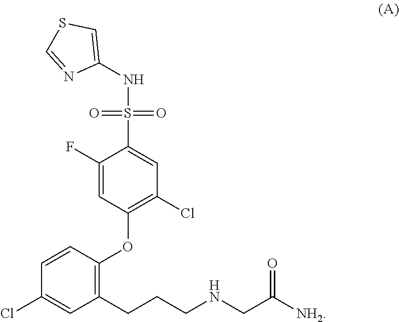 [SMILES: NC(=O)CNCCCc1cc(Cl)ccc1Oc1cc(F)c(S(=O)(=O)Nc2cscn2)cc1Cl]

8. The pharmaceutical ophthalmic formulation according to claim 7, comprising a HCl salt of Compound A

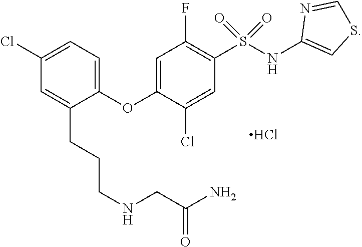 [SMILES: NC(=O)CNCCCc1cc(Cl)ccc1Oc1cc(F)c(S(=O)(=O)Nc2cscn2)cc1Cl ; Cl]

9. A method for treating or alleviating an eye condition comprising administering to a subject in need thereof a therapeutically effective amount of a pharmaceutical ophthalmic formulation according to claim 7, wherein the eye condition is dry eye pain, chronic ocular surface pain, corneal neuralgia, corneal neuropathy, corneal neuropathic pain, chronic post-surgical corneal, ocular and/or scleral pain, Stevens-Johnson Syndrome, Sjogren's Syndrome or any auto-immune disease with ocular pain, moderate to severe primary open-angle glaucoma, corneal abrasion/ocular mechanical injury, contact lens pain, corneal ulcers, a pain caused by infections, ectropion, entropion, acute closed-angle glaucoma, allergic conjunctivitis, blepharitis, intravitreal injections, chalazion, or pain from glaucoma shunt surgery or failed shunt, or an acute ocular injury or a chronic pain associated with dry eye disease.

10. The method according to claim 9, wherein chronic post-surgical corneal, ocular and/or scleral pain comprises LASIK, photorefractive keratectomy, second-eye cataract, corneal transplant, or any other refractive surgeries involving invasive corneal or scleral trauma.

11. The method according to claim 9, wherein the auto-immune disease comprises Stevens-Johnson Syndrome, Sjogren's Syndrome, Grave's Disease, Blau Syndrome, Cogan Syndrome, Leber Congenital Amaurosis, ocular cicatricial pemphigoid, ocular sarcoidosis, Stargardt's disease, or other systemic autoimmune diseases selected from multiple sclerosis, Lupus Erythromatosis, Behcets Disease, and/or Rheumatoid Arthritis.

12. The method according to claim 9, wherein the pain caused by infections comprises bacterial or viral infections leading to iritis, episcleritis, scleritis, conjunctivitis, keratitis, uveitis, or herpetic infection.

13. A pharmaceutical ophthalmic formulation comprising about 15 mg/mL Compound A or a HCl salt of Compound A, about 12% (w/w) HPβCD, about 4% (w/w) polysorbate 80, and about 3% (w/w) HPMC in 20 mM phosphate buffer

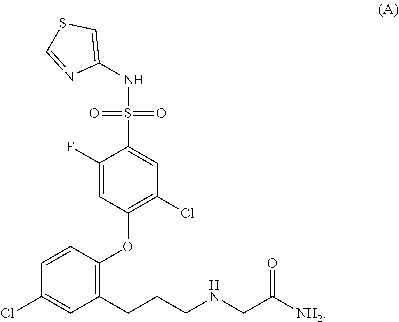 [SMILES: NC(=O)CNCCCc1cc(Cl)ccc1Oc1cc(F)c(S(=O)(=O)Nc2cscn2)cc1Cl]

14. The pharmaceutical ophthalmic formulation according to claim 13, comprising a HCl salt of Compound A

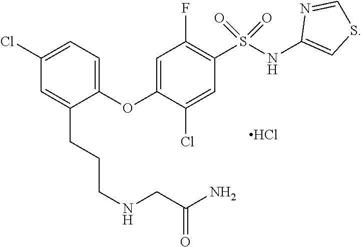 [SMILES: NC(=O)CNCCCc1cc(Cl)ccc1Oc1cc(F)c(S(=O)(=O)Nc2cscn2)cc1Cl ; Cl]

15. A method for treating or alleviating an eye condition comprising administering to a subject in need thereof a therapeutically effective amount of a pharmaceutical ophthalmic formulation according to claim 13, wherein the eye condition is dry eye pain, chronic ocular surface pain, corneal neuralgia, corneal neuropathy, corneal neuropathic pain, chronic post-surgical corneal, ocular and/or scleral pain, Stevens-Johnson Syndrome, Sjogren's Syndrome or any auto-immune disease with ocular pain, moderate to severe primary open-angle glaucoma, corneal abrasion/ocular mechanical injury, contact lens pain, corneal ulcers, a pain caused by infections, ectropion, entropion, acute closed-angle glaucoma, allergic conjunctivitis, blepharitis, intravitreal injections, chalazion, or pain from glaucoma shunt surgery or failed shunt, or an acute ocular injury or a chronic pain associated with dry eye disease.

16. The method according to claim 15, wherein chronic post-surgical corneal, ocular and/or scleral pain comprises LASIK, photorefractive keratectomy, second-eye cataract, corneal transplant, or any other refractive surgeries involving invasive corneal or scleral trauma.

17. The method according to claim 15, wherein the auto-immune disease comprises Stevens-Johnson Syndrome, Sjogren's Syndrome, Grave's Disease, Blau Syndrome, Cogan Syndrome, Leber Congenital Amaurosis, ocular cicatricial pemphigoid, ocular sarcoidosis, Stargardt's disease, or other systemic autoimmune diseases selected from multiple sclerosis, Lupus Erythromatosis, Behcets Disease, and/or Rheumatoid Arthritis.

18. The method according to claim 15, wherein the pain caused by infections comprises bacterial or viral infections leading to iritis, episcleritis, scleritis, conjunctivitis, keratitis, uveitis, or herpetic infection.

19. A pharmaceutical in situ gel formulation comprising (a) about 50 mg/mL Compound A or a HCl salt of Compound A, about 22.5% Poloxamer 407, about 2.1% Poloxamer 188, about 0.5% HPMC in water; or (b) about 50 mg/mL Compound A or a HCl salt of Compound A, about 21.0% Poloxamer 407, about 1.9% Poloxamer 188, about 0.5% HPMC, 0.9% NaCl in water

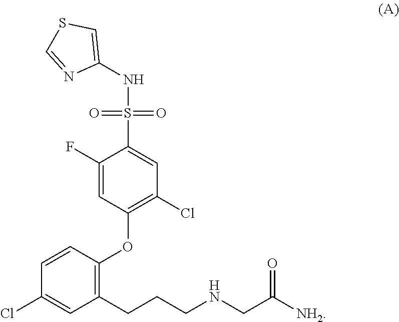 [SMILES: NC(=O)CNCCCc1cc(Cl)ccc1Oc1cc(F)c(S(=O)(=O)Nc2cscn2)cc1Cl]

20. The pharmaceutical in situ gel formulation according to claim 19, comprising a HCl salt of Compound A

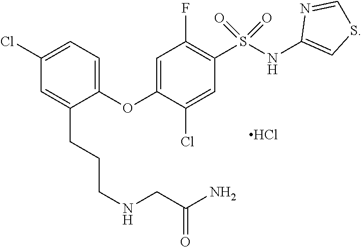 [SMILES: NC(=O)CNCCCc1cc(Cl)ccc1Oc1cc(F)c(S(=O)(=O)Nc2cscn2)cc1Cl ; Cl]

21. A method for treating or alleviating an eye condition comprising administering to a subject in need thereof a therapeutically effective amount of a pharmaceutical ophthalmic formulation according to claim 19, wherein the eye condition is dry eye pain, chronic ocular surface pain, corneal neuralgia, corneal neuropathy, corneal neuropathic pain, chronic post-surgical corneal, ocular and/or scleral pain, Stevens-Johnson Syndrome, Sjogren's Syndrome or any auto-immune disease with ocular pain, moderate to severe primary open-angle glaucoma, corneal abrasion/ocular mechanical injury, contact lens pain, corneal ulcers, a pain caused by infections, ectropion, entropion, acute closed-angle glaucoma, allergic conjunctivitis, blepharitis, intravitreal injections, chalazion, or pain from glaucoma shunt surgery or failed shunt, or an acute ocular injury or a chronic pain associated with dry eye disease.

22. The method according to claim 21, wherein chronic post-surgical corneal, ocular and/or scleral pain comprises LASIK, photorefractive keratectomy, second-eye cataract, corneal transplant, or any other refractive surgeries involving invasive corneal or scleral trauma.

23. The method according to claim 21, wherein the auto-immune disease comprises Stevens-Johnson Syndrome, Sjogren's Syndrome, Grave's Disease, Blau Syndrome, Cogan Syndrome, Leber Congenital Amaurosis, ocular cicatricial pemphigoid, ocular sarcoidosis, Stargardt's disease, or other systemic autoimmune diseases selected from multiple sclerosis, Lupus Erythromatosis, Behcets Disease, and/or Rheumatoid Arthritis.

24. The method according to claim 21, wherein the pain caused by infections comprises bacterial or viral infections leading to iritis, episcleritis, scleritis, conjunctivitis, keratitis, uveitis, or herpetic infection.
